(19) United States
(12) Patent Application Publication
(10) Pub. No.: US 2023/0248866 A1
(43) Pub. Date: Aug. 10, 2023

(54) PORTABLE HYDROXYL ION GENERATOR APPARATUS

(71) Applicant: Turntek, LLC, Greenwood, IN (US)

(72) Inventors: Dustin Scott Jenkins, Indianapolis, IN (US); Christopher Martin Zazzetti, Indianapolis, IN (US); Robert Turner, Indianapolis, IN (US); Brian Williams, Morgantown, IN (US); Madelynn Turner, Indianapolis, IN (US)

(21) Appl. No.: 18/107,776

(22) Filed: Feb. 9, 2023

Related U.S. Application Data
(60) Provisional application No. 63/267,808, filed on Feb. 10, 2022.
(60) Provisional application No. 63/373,743, filed on Aug. 29, 2022.

(51) Int. Cl. A61L 2/22; B01D 53/44; A61L 2/26

(52) U.S. Cl. CPC A61L 2/22; B01D 53/44; A61L 2/26; B01D 2258/06; A61L 2202/16; A61L 2202/11; A61L 2202/15; A61L 2202/14

(57) ABSTRACT

The invention describes a portable apparatus to treat surfaces and the air with hydroxyl ions to reduce the viability and/or kill pathogens.

### FIELD OF THE INVENTION

The invention relates generally to a portable apparatus that generates sufficient hydroxyl ion content to treat and/or kill pathogens, such as allergens, pollen, protozoa, fungi, molds, viruses, bacteria, etc. on the individual, and in the air near the apparatus.

### BACKGROUND OF THE INVENTION

Coronaviruses are a group of related RNA viruses that affect mammals and birds. In humans and birds, such viruses can cause respiratory tract infections that range from mild to lethal. Mild illnesses in humans include some cases of the common cold (which is also caused by other viruses, predominantly rhinoviruses), while more lethal varieties can cause SARS, MERS, and COVID-19 as well as COVID-19 variants. A few vaccines are currently being marketed to treat COVID-19, however, what are termed the “delta” and “omicron” variants appear to be more virulent and are causing some breakthrough cases where the vaccine is not completely effective.

In particular, COVID-19 and variants thereof, such as the delta and mu variants, also referred to as SARS-CoV-2 and the variants thereof, is thought to spread mainly from person to person, mainly through respiratory droplets produced when an infected person coughs or sneezes. These droplets can land in the mouths or noses of people who are nearby or possibly be inhaled into the lungs. It is thought that spread is more likely when people are in close contact with one another (within about 6 feet). It may be possible that a person can get COVID-19 or a variant by touching a surface or object that has the virus on it and then touching their own mouth, nose, or possibly their eyes, however, there is no definitive answer at this time.

An important issue associated with coronaviruses, such as COVID-19 and variants, is how to control the spread of the virus. This aspect is especially important to consider when individuals travel about the world via public transportation, airplanes, trains and the like. Additionally, questions exist as to how to control the spread of the virus in settings where there are often large gatherings of individuals such as schools, restaurants, office buildings, hospitals, clinics, emergency rooms and the like.

Therefore, a need exists for an efficient method and apparatus that can minimize and or eliminate airborne or surface deposited viruses.

### BRIEF SUMMARY OF THE INVENTION

The present embodiments surprisingly provides methods and apparatus' to subject an object, such as an individual, to an atmosphere containing hydroxyl ions to reduce or eliminate unwanted pathogens, such as allergens, pollen, protozoa, fungi, molds, bacteria, or viruses, such as corona viruses.

The present embodiments also provide methods and apparatus' to reduce or eliminate unwanted constituents in the air such as volatile organic components or smoke.

According to one aspect of the disclosed embodiments, an apparatus includes a portable body defining a cavity. The cavity is configured to retain a fluid. An atomizer is positioned in the cavity and in contact with the fluid to produce an atomized fluid. A hydroxyl ion generator produces hydroxyl ions from the atomized fluid. A fan disperses the hydroxyl ions from the portable body.

In some embodiments, a lid of the body can include a plurality of orifices. The hydroxyl ions can be transmitted through the plurality of orifices. The hydroxyl ion generator can include a photocatalyst reactor panel to capture the atomized fluid. The photocatalyst reactor panel can include a perforated mesh. The perforated mesh can be coated with titanium dioxide. The photocatalyst reactor panel can include a pair of spaced apart photocatalyst reactor panels. The hydroxyl ion generator can include a light emitting source to produce the hydroxyl ions. The light emitting source can include a light emitting diode. The light emitting source can include a UV light generator. The light emitting source can provide a spectrum of light from about 320 nm to about 385 nm for UVA and 254 nm to about 275 nm for UVC. The fluid can be water. The fan can disperse the hydroxyl ions within a radius of about 20 feet. A rechargeable battery can be provided to power at least one of the atomizer, the hydroxyl ion generator, and the fan. A hydroxyl concentration of the hydroxyl ions can be at a level that reduces or eliminates one or more pathogens upon exposure to the hydroxyl ions. The fan can disperse the hydroxyl ions into a surrounding area. The portable body can be sized and shaped to be hand-held. The portable body can be sized and shaped to position in a cup holder. The body can include a lower body and an upper body. A sidewall of the body can taper inward from the upper body to the lower body. The lower body can have a diameter that is less than a diameter of the upper body. The lower body can be sized and shaped to position in a cup holder.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 is an exploded view of a portable hydroxyl ion generator apparatus in accordance with an embodiment, wherein a portion of the lower body of the apparatus is cut-away to illustrate a cavity and a fluid within the cavity;

FIG. 2 is a perspective side view of the portable hydroxyl ion generator apparatus;

FIG. 3 is a front elevation view of the portable hydroxyl ion generator apparatus;

FIG. 4 is a rear elevation view of the portable hydroxyl ion generator apparatus;

FIG. 5 is a first side elevation view of the portable hydroxyl ion generator apparatus;

FIG. 6 is a second side elevation view of the portable hydroxyl ion generator apparatus;

FIG. 7 is a schematic view of a control system of the portable hydroxyl ion generator apparatus;

FIG. 8 is a schematic view of a plurality of portable hydroxyl ion generator apparatuses including a master unit and at least one slave unit;

FIG. 9 is a perspective view of a portable hydroxyl ion generator apparatus in accordance with another embodiment;

FIG. 10 is a cross-sectional view of the apparatus shown in FIG. 9 taken along line 10-10;

FIG. 11 is an exploded view of the apparatus shown in FIG. 9;

FIG. 12 is a graph illustrating data from three separate trials conducted on the apparatuses described herein; and

FIG. 13 is an alternative embodiment of an atomizer configured for use with the portable hydroxyl ion generator apparatus of FIGS. 1 and 9.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Currently, the world is dealing with a pandemic due to the spread of COVID-19 and variants thereof. At present, there isn't a drug regime to combat the effects of the virus let alone a particular vaccine that is 100% effective to prevent infection of the virus from person to person. Methods to treat objects that may have been exposed to viruses are somewhat limited in that efficacy of treatment is not fully understood. Constant cleaning of surfaces, face masks, social distancing, avoidance of large groups of people, etc. can only prevent transmission of viruses to a certain extent. Because of this uncertainty, mass transit travel has become limited, visits to hospitals, clinics, and/or emergency rooms have become questionable as to whether a healthy person may be subjected to unwanted viruses, etc. The result has been, and continues to be, a major disruption to businesses, the economy, health and safety considerations, health care, education, etc. The present embodiments provide a portable safe, efficient, and rapid approach to decontaminate surfaces and/or the air. The device described herein is safe and effective in the treatment of bacteria and viruses, such as COVID-19.

It should be understood that throughout this specification, reference to COVID-19 refers to infectious disease caused by the novel coronavirus, SARS-CoV-2, that appeared in late 2019 and also includes variants thereof. COVID-19 is predominantly a respiratory illness that can affect other organs. People with COVID-19 have reported a wide range of symptoms, ranging from mild symptoms to severe illness. Symptoms may appear 2 to 14 days after exposure to the virus. Symptoms may include: fever or chills; cough; shortness of breath; fatigue; muscle and body aches; headache; new loss of taste or smell; sore throat; congestion or runny nose; nausea or vomiting; diarrhea.

COVID-19 variants include, but are not limited to, the Alpha (B.1.1.7), Beta (B.1.351, B.1.351.2, B.1.351.3), Delta (B.1.617.2, AY.1, AY.2, AY.3), Gamma (P.1, P.1.1, P.1.2), Mu (B.1.621), and Omicron and variants of Omicron.

In one aspect, the present embodiments provide an apparatus comprising: a portable body defining a cavity, wherein the cavity is configured to retain a fluid; an atomizer positioned in the cavity and in contact with the fluid to produced atomized fluid; a hydroxyl ion generator to produce hydroxyl ions from the atomized fluid; and a fan to disperse the hydroxyl ions from the portable body.

The hydroxyl ions used with the apparatus described herein are generated by a hydroxyl ion generator. Hydroxyl ion generators are known in the art and include components such as a light emitting source, a photocatalyst reactor panel, and a water source to provide humidity. One or more fans are generally provided to direct the hydroxyl ion airstream from the apparatus.

The light emitting source typically is a UVA or UVC bulb. One or more UV bulbs can be used in the hydroxyl ion generator to obtain maximum formation of hydroxyl ions. Alternatively the light emitting source can be a light emitting diode (LED).

The hydroxyl generator creates hydroxyl radicals through a photocatalytic reaction utilizing UVA or UVC bulbs and at least one photocatalyst reactor panel that typically includes titanium dioxide (TiO₂) coated onto or impregnated into perforated platforms, such as carbon fibers, ceramic panels, aluminum panels and the like. The process utilizes UVA (black light) in the 320 nm to 385 nm region, e.g., 365 nm, or UVC light in the 254 nm to 275 nm region wavelength to excite (irradiate) nano sized titanium dioxide particles. Typical hydroxyl generators generate hydroxyl gas having a hydroxyl concentration of about 900 to about 1000 ppm, in some embodiments.

The hydroxyl radical, •OH, is the neutral form of the hydroxide ion (OH—). Hydroxyl radicals are diatomic molecules that are highly reactive and very short-lived with an average half-life of less than two seconds. Hydroxyls work primarily by abstracting hydrogen atoms, thereby dismantling the molecular structure of volatile organic compounds (VOCs). The chain reaction caused by a cascade of organic oxidizing agents is stable enough to react with nearly all organic chemicals, many inorganic chemicals and smoke (airborne particulates) throughout the entire treatment space. The hydroxyl generator directs hydroxyl ions from the unit to react with the contaminated air where the hydroxyl ions purify the air and prevent pathogens, such as protozoa, fungi, molds, viruses, microorganisms, and other contaminants from multiplying again. As long as the system is in operation, chain reactions continue, ensuring a constant flow of hydroxyl ions. As long as the apparatus is in operation, chain reactions continue, ensuring a constant flow of hydroxyl ions.

The photocatalyst reactor panels can be manufactured from a multitude of materials as noted above. The panels can be a mesh or screen or any support with a porosity (hole size) sufficient to permit maximum airflow through the apparatus. Suitable porous supports include, for example, metallic or porcelain as described above with hole dimensions of from about 0.1 microns to about 5 centimeters. The hole does not need to be symmetrical and can be circular, oblong, trapezoidal, square, etc. Ideally, the photocatalyst reactor panel (coated with titanium dioxide) is configured so as to minimally disrupt the airflow of the system described herein. Alternatively, the photocatalyst reactor panel can be flat or rod sheets/rods of metal.

As noted above, a fan, or a plurality of fans, is utilized to create an airflow to discharge generated hydroxyl ions through from apparatus and disperse the generated hydroxyl ions to a surrounding area. In one embodiment, the fan disperses the generated hydroxyl ions in a radius of about 20 feet in the area surrounding the apparatus. In other embodiments, the fan can disperse the generated hydroxyl ions into a smaller or larger radius.

In operation, the apparatus can provide a hydroxyl concentration of sufficient concentration to eliminate pathogens, including for example, allergens, pollen, protozoa, bacteria, fungi, molds, viruses, etc. (e.g., coronaviruses, such as COVID-19) upon exposure to the hydroxyl ions. It should be understood that the apparatus expels and distributes hydroxyl ions into the air, killing or reducing the ill effects of allergens, pollen, protozoa, viruses, molds, fungi, bacteria, etc. and on surfaces in the areas where the hydroxyl ion is active/reactive.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

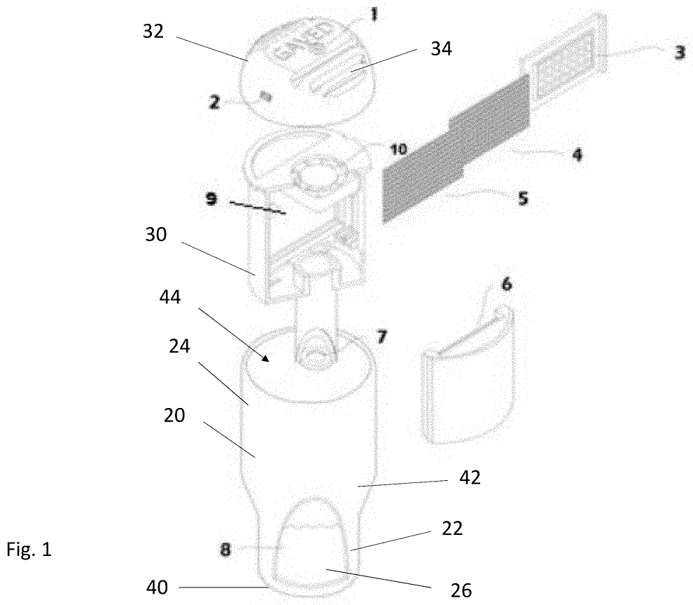

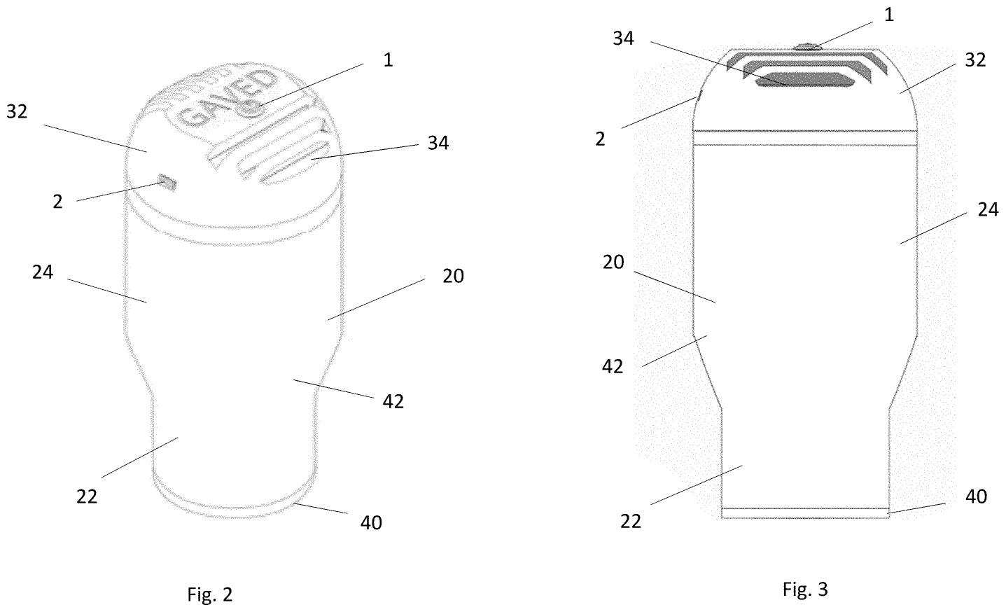

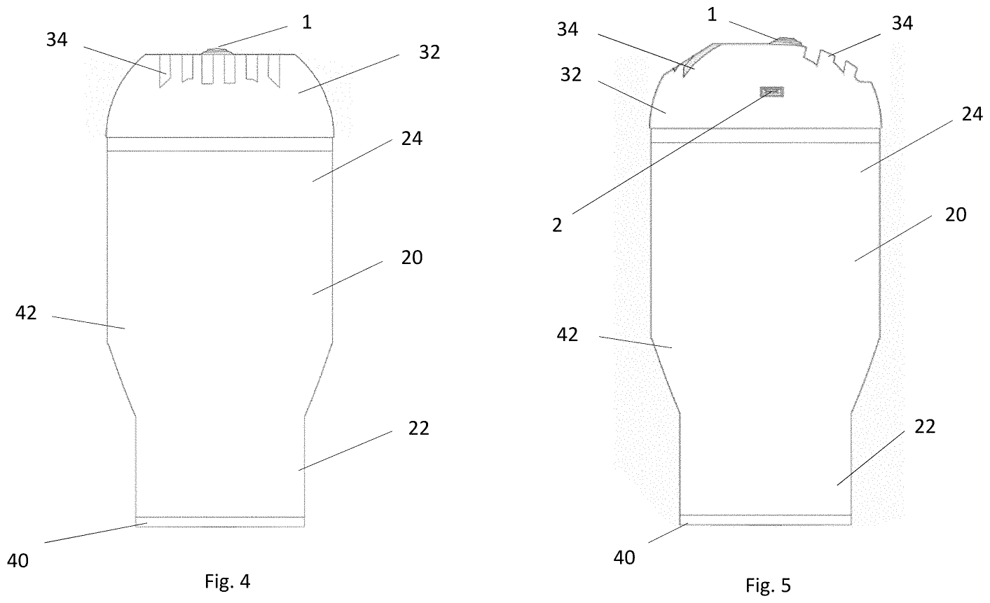

FIGS. 1-8 provide a general embodiment of the portable hydroxyl ion generator apparatus 50 described herein.

The apparatus 50 includes a portable body 20 that is configured to be hand-held. In some embodiments, the portable body 20 is sized and shaped to position in a cup holder, for example, the cup holder of a vehicle. For example, the portable body 20 can have a generally round configuration. It will be appreciated that the portable body 20 can have other suitable shapes. In some embodiments, the portable body 20 is configured to be positioned in a room, for example, on a counter, desk, or the like. The portable body 20 is configured to be moved to any area that is intended to be disinfected.

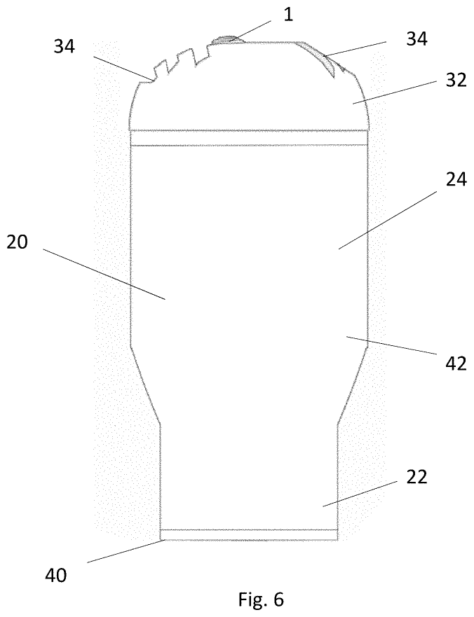

The portable body 20 includes a base 40 and a sidewall 42 extending from the base 40 to an opening 44. In an exemplary embodiment, a height of the portable body 20 is about 9¾ inches. A cavity 8 extends from the opening 44 to the base 40. The cavity 8 is configured to retain a fluid 26 to be atomized. In the exemplary embodiment, the fluid 26 is water. FIG. 6 illustrates the fluid 26 in the cavity 8 through a cut-away in the sidewall 42. It will be appreciated that other fluids capable of providing the disinfecting properties described herein can be utilized with the apparatus.

The portable body 20 includes a lower body 22 and an upper body 24. The lower body 22 extends upward from the base 40 to the upper body 24. The upper body 24 extends upward from the lower body 22 to the opening 44. The sidewall 42 tapers inward from the upper body 24 to the lower body 22 so that the lower body 22 has a diameter that is less than the upper body 24. In an exemplary embodiment, the lower body 22 has a diameter of about 3 inches and the upper body 24 has a diameter of about 4 inches. In some embodiments, the lower body 22 is sized and shaped to position in a cup holder.

A lid 32 is configured to position over the opening 44 on the upper body 24 to seal the cavity 8. The lid 32 includes at least one orifice 34 for the discharge of hydroxyl ions from the apparatus. In the illustrated embodiment, the lid 32 includes a plurality of orifices 34. The orifices 34 face in opposite directions to maximize a discharge radius of the hydroxyl ions from the apparatus. A power switch 1 is provided on the lid 32 to power the apparatus. Additionally, a charging port 2 is provided on the lid 32 to charge the apparatus. It will be appreciated that the design of the apparatus can be altered so that the power switch 1 and the charging port 2 are positioned on the sidewall 42 of the body 20.

An insert 30 is positioned in the cavity 8 under the lid 32. The insert 30 carries an atomizer 7 to produce an atomized fluid and a hydroxyl ion generator to produce hydroxyl ions from the atomized fluid. In one embodiment, the atomizer 7 is an atomization disc. The insert 30 extends into the cavity 8 into the lower body 22 so that the atomizer 7 is positioned near the base 40 of the body 20. The atomizer 7 is positioned in contact with the fluid 26 so that the atomizer 7 produces atomized fluid.

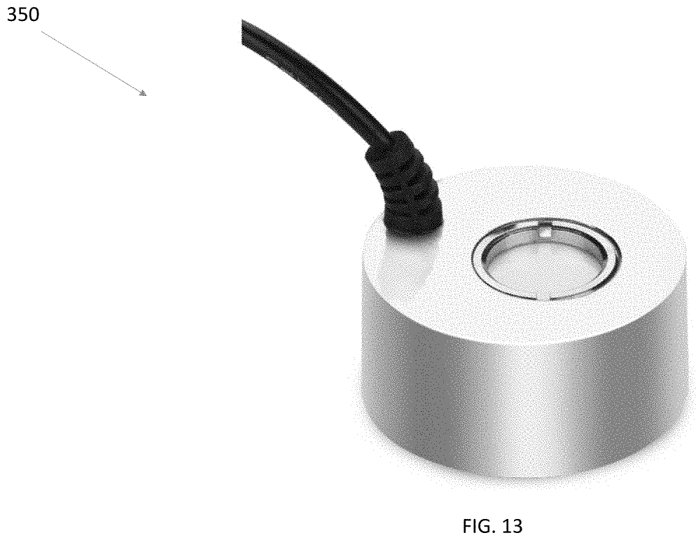

In some embodiments, the atomizer 7 can be replaced with the atomizer 350, shown in FIG. 13. The atomizer 350 is configured to be positioned at the bottom of the cavity 8, in some embodiments. The atomizer 350 uses ultrasonic electronics to convert the water molecules in the cavity 8 into atomized water droplets.

The hydroxyl ion generator includes a rear photocatalyst reactor panel 4 and a front photocatalyst reactor panel 5. The rear photocatalyst reactor panel 4 and the front photocatalyst reactor panel 5 are positioned adjacent one another and spaced apart. Each of the rear photocatalyst reactor panel 4 and the front photocatalyst reactor panel 5 can include a perforated mesh, as described above, to capture the atomized fluid produced by the atomizer 7. In some embodiments, the perforated mesh is coated with titanium dioxide.

The rear photocatalyst reactor panel 4 and the front photocatalyst reactor panel 5 are positioned between a back light panel 3 and a front light panel 6. The back light panel 3 and the front light panel 6 provide a spectrum of light from about 320 nm to about 385 nm for UVA and 254 nm to about 275 nm for UVC onto the rear photocatalyst reactor panel 4 and the front photocatalyst reactor panel 5 to produce the hydroxyl ions from the atomized fluid captured on the rear photocatalyst reactor panel 4 and the front photocatalyst reactor panel 5. The back light panel 3 and the front light panel 6 can include a light emitting diode or a plurality of light emitting diodes. In other embodiments, the back light panel 3 and the front light panel 6 include a UV light generator.

At least one fan 10 is positioned at a top of the insert 30 and adjacent the orifices 34 in the lid 32. The fan 10 draws the hydroxyl ions upward from the cavity 8 and discharges the hydroxyl ions through the orifices 34. The fan 10 disperses the hydroxyl ions into a surrounding area around the apparatus. In some embodiments, the fan 10 disperses the hydroxyl ions within a radius of about 20 feet. It will be appreciated that the radius within which the hydroxyl ions is dispersed can be increased or decreased by altering a speed and/or size of the fan 10. A hydroxyl concentration of the hydroxyl ions dispersed is at a level that reduces or eliminates one or more pathogens upon exposure to the hydroxyl ions.

A rechargeable battery 9 is also positioned in the insert to provide power to the apparatus when the power switch 1 is activated. In some embodiments, the rechargeable battery 9 is a lithium ion battery. The rechargeable battery 9 can be recharged through the charging port 2. It will be appreciated that the rechargeable battery 9 powers at least one of the atomizer, the hydroxyl ion generator, and the fan.

In some embodiments, the apparatus 50 does not include the battery 9. In such embodiments, the apparatus 50 can include an electrical plug suitable for connecting to an outlet. For example, the apparatus 50 can be configured for connecting to a 110 volt ground fault circuit interrupter (GFCI) or other standard household outlet. It will be appreciated that the electrical plug can be adapted for any particular geographic region. It will also be appreciated that the plug can be adapted to connect to an outlet having any suitable voltage.

In another embodiment, the apparatus 50 can include a universal serial bus (USB) charger, a lighting cable, a high-definition multimedia interface (HDMI) cable, or any other suitable changing cable. As such, the apparatus 50 can be charged by a computer or other electronic device. Moreover, the apparatus 50 can be charged by a vehicle having a suitable charging outlet. It will be appreciated that, in some embodiments, the apparatus 50 includes an electrical plug for use with a 12 volt automotive outlet. In some embodiments, the apparatus 50 includes a plurality of plugs that interchangeably insert into the charging port 2.

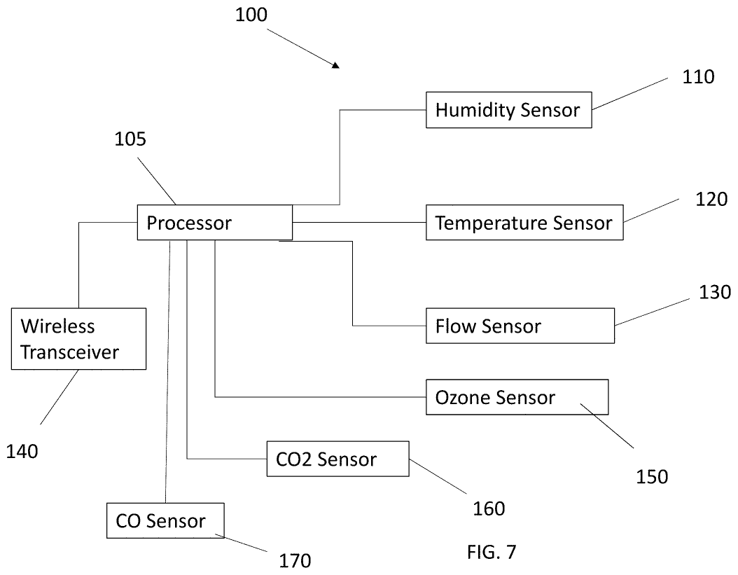

Any of the embodiments described herein can include a control system 100. A schematic of the control system 100 is illustrated in FIG. 7, which will be described with respect to the apparatus 50. The control system 100 includes a processor 105. A humidity sensor 110 monitors a humidity of the hydroxyl ions emitted from the portable body 20. In some embodiments, another humidity sensor can measure a humidity of the airflow into the portable body 20. That is, the humidity sensor 110 can include an inlet humidity sensor 110 and an outlet humidity sensor 110. A temperature sensor 120 monitors a temperature of the hydroxyl ions emitted from the portable body 20. In some embodiments, another temperature sensor can measure a temperature of the airflow into the portable body 20. That is, the temperature sensor 120 can include an inlet temperature sensor 120 and an outlet temperature sensor 120. A flow sensor 130 monitors a flow rate of the hydroxyl ions emitted from the portable body 20. In some embodiments, another flow sensor can measure a flow rate of the airflow into the portable body 20. That is, the flow sensor 130 can include an inlet flow sensor 130 and an outlet flow sensor 130.

The sensors 110, 120, 130 provide feedback to the processor 105 so that the control system 100 can alter the function (e.g. activate and deactivate) of the atomizer 7 and/or the hydroxyl ion generator. At least one of the humidity sensor 110, the temperature sensor 120, and the flow sensor 130 can be located in the portable body 20. In other embodiments, at least one of the humidity sensor 110, the temperature sensor 120, and the flow sensor 130 can be located remotely from the portable body 20. At least one of the humidity sensor 110, the temperature sensor 120, and the flow sensor 130 can be hardwired or wirelessly coupled to the processor 105.

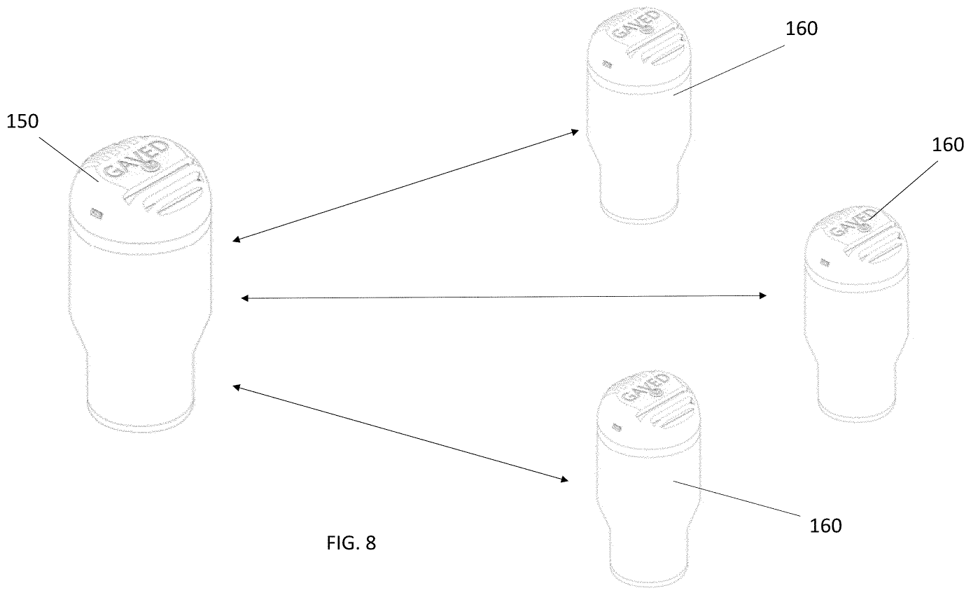

The control system 100 also includes a wireless transceiver 140 that enables the apparatus 50 to wirelessly communicate with other apparatuses 50, as described in more detail below with regard to FIG. 8. In one embodiment, the wireless transceiver 140 is a Bluetooth® device. The wireless transceiver 140 can also enable communication with a remote device, for example, a mobile phone or tablet. Accordingly, the apparatus 50 can be controlled remotely from the remote device. For example, a speed of the fan 10 can be adjusted using the remote device or the fan 10 may be set in a silent mode that reduces an amount of sound produced by the apparatus 50.

In one embodiment, the control system 100 can adjust the flow rate of the hydroxyl ions based on at least one of the humidity as measured by the humidity sensor 110, the temperature as measured by the temperature sensor 120, and the flow rate as measured by the flow sensor 130. In another embodiment, the control system 100 can adjust the flow rate of the hydroxyl ions based on at least two of the humidity as measured by the humidity sensor 110, the temperature as measured by the temperature sensor 120, and the flow rate as measured by the flow sensor 130. In yet another embodiment, the control system 100 can adjust the flow rate of the hydroxyl ions based on all three of the humidity as measured by the humidity sensor 110, the temperature as measured by the temperature sensor 120, and the flow rate as measured by the flow sensor 130. In some embodiments, the flow rate of hydroxyl ions is adjusted by controlling the atomizer 7 (for example, turning the atomizer 7 on and off).

In one embodiment, the control system 100 can adjust the hydroxyl ion generator based on at least one of the temperature as measured by the temperature sensor 120 and the flow rate as measured by the flow sensor 130. In another embodiment, the control system 100 can adjust the hydroxyl ion generator based on both of the temperature as measured by the temperature sensor 120 and the flow rate as measured by the flow sensor 130. In some embodiments, the lights 3 and 6 are controlled (for example, turned on or off) by the control system 100 to control the hydroxyl ion generator.

In some embodiments, the apparatus shuts off at approximately 48% humidity in the enclosed space. In some embodiments, the humidity is maintained within a range of approximately 40% to 60%. In some embodiments, the humidity is maintained within a range of approximately 45%-55%. In some embodiments, the humidity is maintained within a range of approximately 57%-59%. In some embodiments, the humidity is maintained below 60%.

The control system 100 also includes an ozone sensor 150 to measure an amount of ozone produced if by the apparatus 50 or by another apparatus not associated with apparatus 50. The control system 100 can shut off the apparatus 50 if a predetermined level of ozone is detected by the ozone sensor 150. It will be appreciated that in an exemplary embodiment, the hydroxyl ions are produced without producing ozone. The control system 100 can also include a carbon dioxide sensor 160 to measure an amount of carbon dioxide produced by the apparatus 50. The control system 100 can shut off the apparatus 50 if a predetermined level of carbon dioxide is detected by the carbon dioxide sensor 160. The control system 100 can further include a carbon monoxide sensor 170 to measure an amount of carbon monoxide produced by the apparatus 50. The control system 100 can shut off the apparatus 50 if a predetermined level of carbon monoxide is detected by the carbon monoxide sensor 170.

One advantage of the apparatus', systems and methods described herein is that ozone is not generated.

Another advantage of the apparatus', systems and methods described herein is that the hydroxyl ions, once emitted from the apparatus/system will interact with undesired contaminants in the air (e.g., allergens, pollen, protozoa, fungi, bacteria, viruses, etc.) and cause them to decompose and/or degrade so as to not infect an individual or reduce the discomfort that the individual suffers from allergies. Thus the apparatus' and systems can be used to treat such contaminants, reducing and/or eliminating such contaminants.

Still another advantage of the apparatus', systems and methods described herein provides a clean smelling environment that is akin to fresh mountain air.

FIG. 8 illustrates a plurality of apparatuses 50 in communication with one another. The plurality of apparatuses 50 includes a master unit 150 and at least one slave unit 160. In an exemplary embodiment, the master unit 150 controls the at least one slave unit 160. In some embodiments, each of the slave units 160 includes at least one of the sensors 110, 120, 130 described above. The master unit 150 can also include at least one of the sensors 110, 120, 130 described above. The processor 105 can be housed at the master unit 150. Signals from each of the sensors 110, 120, 130 are transmitted to the master unit 150, so that the master unit 150 can individually control each slave unit 160. That is, the master unit 150 can individually control a fan 10 or hydroxyl ion generator of each slave unit 160. In some embodiments, the master unit 150 can individually turn the slave units on and off.

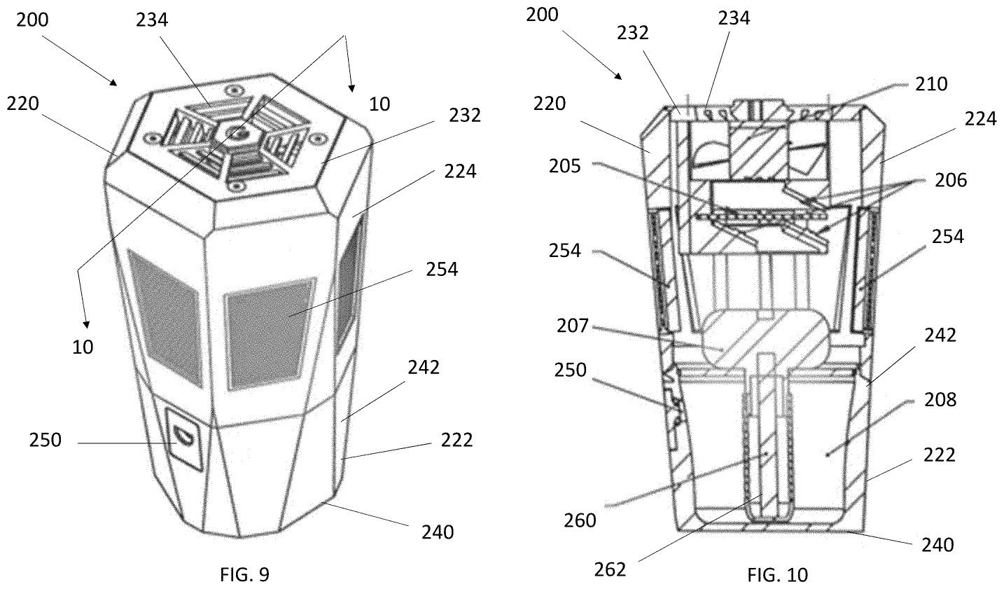

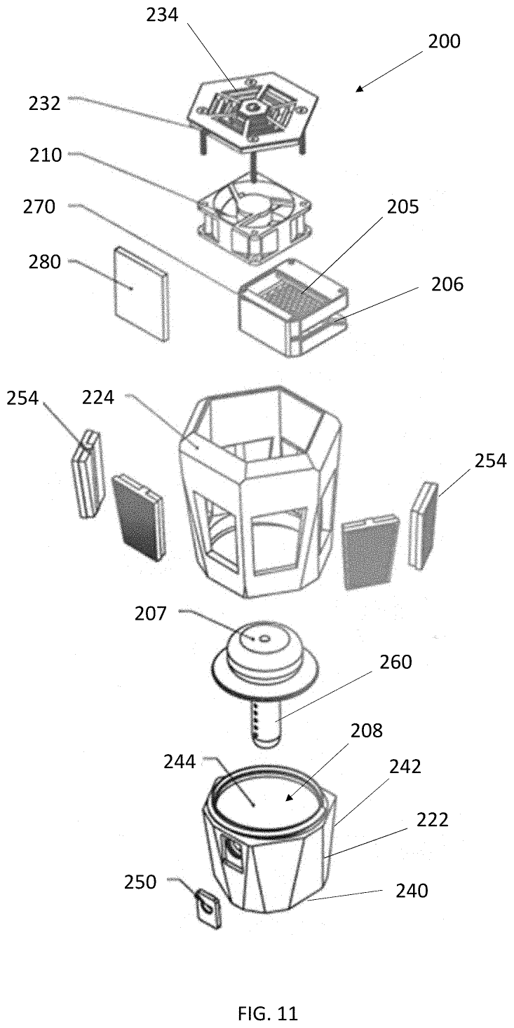

FIGS. 9-11 provide a general embodiment of another portable hydroxyl ion generator apparatus 200 described herein. It will be appreciated the the apparatus 200 can include the control system 100 described in FIG. 7. Additionally, a plurality of apparatuses 200 can operate in a master/slave relationship as described in FIG. 8.

The apparatus 200 includes a portable body 220 that is configured to be hand-held. In some embodiments, the portable body 220 is sized and shaped to position in a cup holder, for example, the cup holder of a vehicle. In some embodiments, the portable body 220 is configured to be positioned in a room, for example, on a counter, desk, or the like. The portable body 220 is configured to be moved to any area that is intended to be disinfected.

The portable body 220 includes a base 240 and a sidewall 242 extending from the base 240 to an opening 244. In an exemplary embodiment, a height of the portable body 220 is about 9 3/4 inches. A cavity 208 extends from the opening 244 to the base 240. The cavity 208 is configured to retain a fluid to be atomized. In the exemplary embodiment, the fluid is water. It will be appreciated that other fluids capable of providing the disinfecting properties described herein can be utilized with the apparatus.

The portable body 220 includes a lower body 222 and an upper body 224. The lower body 222 extends upward from the base 240 to the upper body 224. The upper body 224 extends upward from the lower body 222 to the opening 244. The lower body 222 includes a fluid fill lid 250 that can be opened to access the cavity 208 so that fluid can be poured into the cavity 208. The upper body 224 includes at least one filter 254 that filter the air being drawn into the apparatus 200. A lid 232 is configured to position on the upper body 224. The lid 232 includes at least one orifice 234 for the discharge of hydroxyl ions from the apparatus. In the illustrated embodiment, the lid 232 includes a plurality of orifices 234. The orifices 234 face in opposite directions to maximize a discharge radius of the hydroxyl ions from the apparatus.

An atomizer 207 is provided to produce an atomized fluid. In one embodiment, the atomizer 207 is an atomization disc. A fluid transport device 260 is configured to moves the fluid in the cavity 208 into contact with the atomizer 207. In the exemplary embodiment, the fluid transport device 260 includes a wick 262 extending from the atomizer 207 into the cavity 208. The wick 262 can be formed from cotton or any other absorbable material. In some embodiments, the wick 262 acts as a filter to filter to fluid coming in contact with the atomizer 207. The wick 262 can be replaceable. In an alternative embodiment, the fluid transport device 260 sprays the fluid onto the atomizer. In another embodiment, the fluid transport device 260 includes a pump.

In some embodiments, the atomizer 207 can be replaced with the atomizer 350, shown in FIG. 13. The atomizer 350 is configured to be positioned at the bottom of the cavity 208, in some embodiments. The atomizer 350 uses ultrasonic electronics to convert the water molecules in the cavity 208 into atomized water droplets.

A hydroxyl ion generator 270 includes at least one photocatalyst reactor panel 205. The at least one photocatalyst reactor panel 205 is positioned in the upper body 224 above the atomizer 207. The at least one photocatalyst reactor panel 205 can include a perforated mesh, as described above, to capture the atomized fluid produced by the atomizer 207. In some embodiments, the perforated mesh is coated with titanium dioxide.

The hydroxyl ion generator 270 also includes at least one light 206 to provide a spectrum of light from about 320 nm to about 385 nm for UVA and 254 nm to about 275 nm for UVC onto the photocatalyst reactor panel 205 to produce the hydroxyl ions from photocatalyst reactor panel 205. The light 206 can include a light emitting diode or a plurality of light emitting diodes. In other embodiments, the light 206 includes a UV light generator.

At least one fan 210 is positioned at a top of the upper body 224 and adjacent the orifices 234 in the lid 232. The fan 210 draws the hydroxyl ions upward from the cavity 208 and discharges the hydroxyl ions through the orifices 234. The fan 210 disperses the hydroxyl ions into a surrounding area around the apparatus. In some embodiments, the fan 210 disperses the hydroxyl ions within a radius of about 20 feet. It will be appreciated that the radius within which the hydroxyl ions is dispersed can be increased or decreased by altering a speed and/or size of the fan 10. A hydroxyl concentration of the hydroxyl ions dispersed is at a level that reduces or eliminates one or more pathogens upon exposure to the hydroxyl ions.

A control panel 280 is provided to power and control the apparatus 200. The control panel 280 can include the control circuitry 100 described above. The apparatus 200 can include a rechargeable battery, as described above, to provide power to the apparatus 200 when a power switch is activated. In some embodiments, the apparatus 200 includes an electrical plug suitable for connecting to an outlet, as described above. The apparatus 200 can also include a universal serial bus (USB) charger, a lighting cable, a high-definition multimedia interface (HDMI) cable, or any other suitable changing cable, as described above. In some embodiments, an indicator is provided to notify a user when a fluid level in the cavity 208 is low. The indicator can be an audible or visual alert, for example an alarm (beep) or a light.

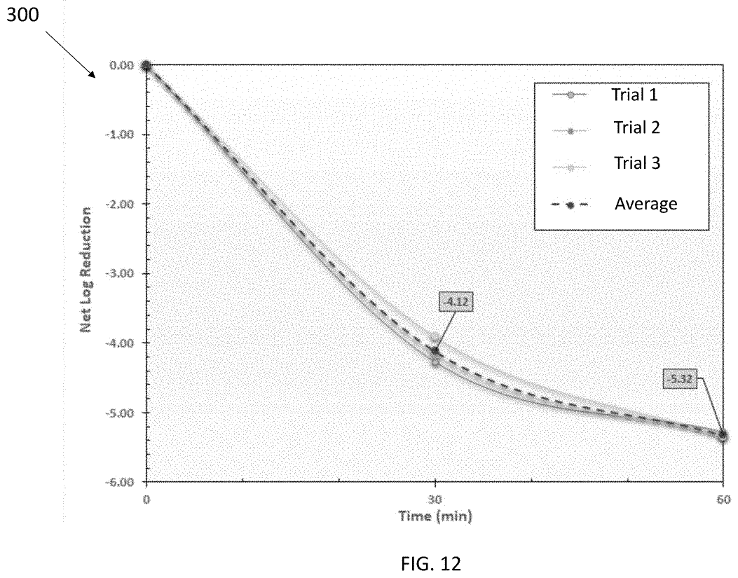

Referring now to FIG. 12, the graph 300 illustrates the results of testing performed on at least one of the apparatuses described herein.

Species selection was based on Biological Safety Level 1 (BSL1) surrogates for BSL2-BSL3 pathogenic organisms. MS2 bacteriophage (ATCC 15597-B1) is an un-enveloped, tail-less virus, that is 25 nm in size. It contains linear ssRNA as its genome and has historically been used as an Influenza surrogate. MS2 has similar aerosol properties to other viruses due to their small size and shape. Measuring the capture efficiency of the Poppy device with MS2 helps create a reliable in-vitro test model. It also may help as an in vitro indicator for how pathogenic viruses could be handled in real world settings.

A 6-Jet Collison nebulizer was used to generate bioaerosol within the chamber. The nebulizer fed bio-aerosol into the chamber through a tri-clover stainless pass-through port centrally located on the back-chamber wall. The system was constructed using stainless sanitary tubing and connected to a common stainless manifold with a controllable dilution air valve. In order to sample the air within the chamber, four AGI-30 impingers (Ace Glass Inc. Vineland N.J.) were used for bioaerosol collection to determine challenge concentrations. These impingers were connected to the test chamber via ⅜″ stainless tube sample ports. These ports were fastened to the chamber using ⅜″ stainless bulkhead fittings. Impingers were filled with 20 mL of sterilized phosphate buffer solution (PBS) containing 0.005% v/v of Tween 80 for bioaerosol collection. These impingers were specifically designed to collect chamber air at a constant flow rate. This allows for precise back calculations of chamber concentration based on the impinger sampling duration. The nebulization air flow rate of the Collison 6-jet nebulizer and dilution air flow rate was controlled and monitored to ensure that the same rates for all of the challenge trials were properly maintained. The Collison 6-jet nebulizer was operated at a head pressure of 35 psi with an approximate output rate of 15 L/min with an additional 85 L/min of dilution air, totaling 100 L/min for all of the trials conducted. All exposures were conducted at room temperature with the internal ceramic unit inside the test chamber set to 20° C. in order to maintain a steady exposure temperature for the duration of the trials. These trials also had humidity regulation to maintain the chamber at approximately 60% relative humidity. All four mixing fans were turned on for the duration of each trial in order to ensure adequate bioaerosol mixing.

Following each chamber characterization trial, the impinger samples were pooled for an overall average of the entire chamber concentration. Using calibrated pippettes, impinger samples were serially diluted and subsequently plated in triplicate for each dilution. These dilutions were plated using a standard small drop assay in triplicate on labeled sterile Tryptic Soy Agar Dishes (Hardy Diagnostics, Santa Maria, Calif.). Impinger samples were then incubated for approximately 24 hours at 37° C.

The results shown in graph 300 represent the total Net Log reduction of MS2 overtime within the testing chamber. The net log reduction considers the control losses determined by the control tests, or tests without the test device operational, and subtracts that result from the log reduction observed during the trials with the device operational. Doing this allows for a clear representation of the device's efficacy without the contribution of natural die off of the challenge organism. Based on the triplicate trials performed to assess the apparatus's ability to remove viable virus from a given area, we've demonstrated an average reduction of 4.12 in just 30 min of device operation. In addition, at 60 minutes the Net log reduction had improved to 5.32. The 4.12 Net log reduction and the 5.32 Net log reduction equate to over a 99.9919% and 99.9995% reduction of total viable virus respectively. These results suggest that the apparatus would effectively remove 99.99% of similar viruses from the air in just 30 minutes of operation in a similarly sized space.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

The following clauses enumerated consecutively from 1 through 58 provide for various aspects of the present invention. In one embodiment, in a first clause (1), the present invention provides an apparatus comprising:

a portable body defining a cavity, wherein the cavity is configured to retain a fluid;

an atomizer positioned in the cavity and in contact with the fluid to produce an atomized fluid;

a hydroxyl ion generator to produce hydroxyl ions from the atomized fluid; and

a fan to disperse the hydroxyl ions from the portable body.

2. The apparatus of clause 1, wherein a lid of the body further comprises a plurality of orifices, wherein the hydroxyl ions can be transmitted through the plurality of orifices.
3. The apparatus of either clause 1 or 2, wherein the hydroxyl ion generator comprises a photocatalyst reactor panel to capture the atomized fluid.
4. The apparatus of clause 3, wherein the photocatalyst reactor panel comprises a perforated mesh.
5. The apparatus of clause 4, wherein the perforated mesh is coated with titanium dioxide.
6. The apparatus of clause 3, wherein the photocatalyst reactor panel includes a pair of spaced apart photocatalyst reactor panels.
7. The apparatus of any of clauses 1 through 6, wherein the hydroxyl ion generator comprises a light emitting source to produce the hydroxyl ions.
8. The apparatus of clause 7, wherein the light emitting source comprises a light emitting diode.
9. The apparatus clause 7, wherein the light emitting source comprises a UV light generator.
10. The apparatus of clause 7, wherein the light emitting source provides a spectrum of light from about 320 nm to about 385 nm for UVA and 254 nm to 275 nm for UVC.
11. The apparatus of any of clauses 1 through 10, wherein the fluid is water.
12. The apparatus of any of clauses 1 through 11, wherein the fan disperses the hydroxyl ions within a radius of about 20 feet.
13. The apparatus of any of clauses 1 through 12, further comprising a rechargeable battery to power at least one of the atomizer, the hydroxyl ion generator, and the fan.
14. The apparatus of any of clauses 1 through 13, wherein a hydroxyl concentration of the hydroxyl ions is at a level that reduces or eliminates one or more pathogens upon exposure to the hydroxyl ions.
15. The apparatus of any of clauses 1 through 14, wherein the fan disperses the hydroxyl ions into a surrounding area.
16. The apparatus of any of clauses 1 through 15, wherein the portable body is sized and shaped to be hand-held.
17. The apparatus of any of clauses 1 through 16, wherein the portable body is sized and shaped to position in a cup holder.
18. The apparatus of any of clauses 1 through 17, wherein the body includes a lower body and an upper body, wherein a sidewall of the body tapers inward from the upper body to the lower body.
19. The apparatus of clause 18, wherein the lower body has diameter that is less than a diameter of the upper body.
20. The apparatus of clause 18, wherein the lower body is sized and shaped to position in a cup holder.
21. The apparatus of any of clauses 1 through 20, further comprising a control system having:

a humidity sensor to monitor a humidity discharged from the portable body,

a temperature sensor to monitor a temperature of the hydroxyl ions, and

a flow sensor to monitor a flow rate of the hydroxyl ions.

22. The apparatus of clause 21, wherein the control system adjusts the atomizer based on the humidity as measured by the humidity sensor.
23. The apparatus of clause 21, wherein the control system adjusts the atomizer based on the temperature as measured by the temperature sensor.
24. The apparatus of clause 21, wherein the control system adjusts the atomizer based on the flow rate as measured by the flow sensor.
25. The apparatus of clause 21, wherein the control system adjusts the atomizer based on a combination of at least two of the humidity as measured by the humidity sensor, the temperature as measured by the temperature sensor, and the flow rate as measured by the flow sensor.
26. The apparatus of clause 21, wherein the control system adjusts a light of the hydroxyl ion generator based on the temperature as measured by the temperature sensor.
27. The apparatus of clause 21, wherein the control system adjusts a light of the hydroxyl ion generator based on the flow rate as measured by the flow sensor.
28. The apparatus of clause 21, wherein the control system adjusts a light of the hydroxyl ion generator based on a combination of the temperature as measured by the temperature sensor and the flow rate as measured by the flow sensor.
29. The apparatus of clause 21, wherein the control system controls the hydroxyl ion generator based on the temperature as measured by the temperature sensor.
30. The apparatus of clause 21, wherein the control system controls the hydroxyl ion generator based on the flow rate as measured by the flow sensor.
31. The apparatus of clause 21, wherein the control system controls the hydroxyl ion generator based on a combination of the temperature as measured by the temperature sensor and the flow rate as measured by the flow sensor.
32. The apparatus of clause 21, wherein the control system maintains a humidity within a range of 40% to 60%.
33. The apparatus of clause 21, wherein the control system maintains a humidity within a range of 57% to 59%.
34. The apparatus of clause 21, wherein the control system maintains a humidity below 60%.
35. The apparatus of clause 21, wherein the control system includes a wireless transceiver, wherein the apparatus is remotely controllable by a remote device through the wireless transceiver.
36. The apparatus of clause 21, wherein the humidity sensor includes:

an inlet humidity sensor positioned at an air intake of the portable body, and

an outlet humidity sensor positioned at an outlet of the portable body.

37. The apparatus of clause 21, wherein the temperature sensor includes:

an inlet temperature sensor positioned at an air intake of the portable body, and an outlet temperature sensor positioned at an outlet of the portable body.

38. The apparatus of clause 21, wherein the flow rate sensor includes:

an inlet flow rate sensor positioned at an air intake of the portable body, and

an outlet flow rate sensor positioned at an outlet of the portable body.

39. The apparatus of any of clauses 1 through 38, further comprising an ozone sensor, wherein the apparatus is inactivated if a predetermined level of ozone is detected by the ozone sensor.
40. The apparatus of any of clauses 1 through 39, further comprising a carbon dioxide sensor, wherein the apparatus is inactivated if a predetermined level of carbon dioxide is detected by the carbon dioxide sensor.
41. The apparatus of any of clauses 1 through 40, further comprising a carbon monoxide sensor, wherein the apparatus is inactivated if a predetermined level of carbon monoxide is detected by the carbon monoxide sensor.
42. The apparatus of any of clauses 1 through 41, further comprising a plurality of portable bodies configured to emit the hydroxyl ions.
43. The apparatus of clause 42, wherein the plurality of portable bodies includes:

a master portable body, and

at least one slave portable body that is controlled by the master directional airflow apparatus.

44. The apparatus of clause 42, wherein each of the plurality of portable bodies includes a wireless transceiver, wherein the plurality of portable bodies communicate through the wireless transceiver.
45. The apparatus of any of clauses 1 through 44, further comprising a fluid transport device configured to move the fluid into contact with the atomizer.
46. The apparatus of clause 45, wherein the fluid transport device includes a wick extending into the cavity.
47. The apparatus of clause 46, wherein the wick is formed from cotton.
48. The apparatus of clause 46, wherein the wick is replaceable.
49. The apparatus of clause 46, wherein the wick filters the fluid.
50. The apparatus of clause 45, wherein the fluid transport device sprays the fluid onto the atomizer.
51. The apparatus of clause 45, wherein the fluid transport device includes a pump.
52. The apparatus of any of clauses 1 through 51, further comprising an indicator to provide an alert when the fluid in the cavity is at a predetermined level.
53. A method to reduce or eliminate a pathogen on a surface or in the air comprising the step of subjecting air to hydroxyl ions generated by an apparatus of any of clauses 1 through 52, wherein the presence of the pathogen is reduced or eliminated.
54. The method of clause 53, wherein the pathogen is an allergen, pollen, protozoa, fungi, molds, viruses, bacteria or mixtures thereof.
55. A method to decompose or degrade a pathogen on a surface or in the air comprising the step of subjecting air to hydroxyl ions generated by an apparatus of any of clauses 1 through 52, wherein the presence of the pathogen is decomposed or degraded.
56. The method of clause 55, wherein the pathogen is an allergen, pollen, protozoa, fungi, molds, viruses, bacteria or mixtures thereof.
57. A method to reduce or eliminate one or more volatile organic components or particulates in the air comprising the step of subjecting air with one or more volatile organic component or particulates to hydroxyl ions generated by an apparatus of any of clauses 1 through 52, wherein the presence of the one or more volatile organic component or particulates are reduced or eliminated from the air.
58. A method to at least one of reduce, eliminate, decompose or degrade a pathogen or one or more volatile organic components or particulates on a surface or in the air, the method comprising the step of subjecting air to hydroxyl ions generated by an apparatus comprising:

a portable body defining a cavity, wherein the cavity is configured to retain a fluid;

an atomizer positioned in the cavity and in contact with the fluid to produce an atomized fluid;

a hydroxyl ion generator to produce the hydroxyl ions from the atomized fluid; and

a fan to disperse the hydroxyl ions from the portable body.

What is claimed is:

1. An apparatus comprising:
a portable body defining a cavity, wherein the cavity is configured to retain a fluid;
an atomizer positioned in the cavity and in contact with the fluid to produce an atomized fluid;
a hydroxyl ion generator to produce hydroxyl ions from the atomized fluid; and
a fan to disperse the hydroxyl ions from the portable body.

2. The apparatus of claim 1, wherein a lid of the body further comprises a plurality of orifices, wherein the hydroxyl ions can be transmitted through the plurality of orifices.

3. The apparatus of claim 1, wherein the hydroxyl ion generator comprises a photocatalyst reactor panel to capture the atomized fluid.

4. The apparatus of claim 3, wherein the photocatalyst reactor panel comprises a perforated mesh.

5. The apparatus of claim 4, wherein the perforated mesh is coated with titanium dioxide.

6. The apparatus of claim 3, wherein the photocatalyst reactor panel includes a pair of spaced apart photocatalyst reactor panels.

7. The apparatus of claim 1, wherein the hydroxyl ion generator comprises a light emitting source to produce the hydroxyl ions.

8. The apparatus of claim 7, wherein the light emitting source comprises at least one of a light emitting diode and a UV light generator.

9. The apparatus of claim 7, wherein the light emitting source provides a spectrum of light from about 320 nm to about 385 nm for UVA and 254 nm to 275 nm for UVC.

10. The apparatus of claim 1, further comprising a rechargeable battery to power at least one of the atomizer, the hydroxyl ion generator, and the fan.

11. The apparatus of claim 1, wherein a hydroxyl concentration of the hydroxyl ions is at a level that reduces or eliminates one or more pathogens upon exposure to the hydroxyl ions.

12. The apparatus of claim 1, wherein the fan disperses the hydroxyl ions into a surrounding area.

13. The apparatus of claim 1, wherein the portable body is sized and shaped to at least one of be hand-held and position in a cup holder.

14. The apparatus of claim 1, wherein the body includes a lower body and an upper body, wherein a sidewall of the body tapers inward from the upper body to the lower body.

15. The apparatus of claim 14, wherein the lower body is sized and shaped to position in a cup holder.

16. An apparatus comprising:
a portable body defining a cavity, wherein the cavity is configured to retain a fluid;
an atomizer positioned in the cavity and in contact with the fluid to produce an atomized fluid;
a hydroxyl ion generator to produce hydroxyl ions from the atomized fluid;
a fan to disperse the hydroxyl ions from the portable body; and
a sensor to monitor a property of the hydroxyl ions.

17. The apparatus of claim 16, further comprising a control system to adjust at least one of the atomizer, the hydroxyl ion generator, and the fan based on feedback from the sensor.

18. The apparatus of claim 16, wherein the sensor is at least one of:
a humidity sensor to monitor a humidity discharged from the portable body,
a temperature sensor to monitor a temperature of the hydroxyl ions, and
a flow sensor to monitor a flow rate of the hydroxyl ions.

19. The apparatus of claim 16, wherein the sensor is at least one of an ozone sensor, a carbon dioxide sensor, and a carbon monoxide sensor.

20. A method to at least one of reduce, eliminate, decompose or degrade a pathogen or one or more volatile organic components or particulates on a surface or in the air, the method comprising the step of subjecting air to hydroxyl ions generated by an apparatus comprising:
a portable body defining a cavity, wherein the cavity is configured to retain a fluid;
an atomizer positioned in the cavity and in contact with the fluid to produce an atomized fluid;
a hydroxyl ion generator to produce the hydroxyl ions from the atomized fluid; and
a fan to disperse the hydroxyl ions from the portable body.
